(19) United States
(12) Patent Application Publication
(10) Pub. No.: US 2010/0112556 A1
(43) Pub. Date: May 6, 2010

(54) METHOD FOR SAMPLE ANALYSIS USING Q PROBES

(76) Inventors: JEFFREY R. SAMPSON, Loveland, CO (US); Brian J. Peter, Loveland, CO (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 12/264,091

(22) Filed: Nov. 3, 2008

(51) Int. Cl. C12Q 1/68

(52) U.S. Cl. 435/6

(57) ABSTRACT

A method of sample analysis is provided. In certain embodiments, the method may comprise: a) contacting a plurality of Q probes with a nucleic acid sample comprising a target polynucleotide under hybridization conditions to form a plurality of flap endonuclease substrates each comprising a Q probe and a site in the target polynucleotide; b) contacting the plurality of flap endonuclease substrates with a flap endonuclease under cleavage conditions to produce cleavage products, in which each of the Q probes of the flap endonuclease substrates is cleaved to produce cleavage products that include at least a first fragment that is hybridized with a site in the target polynucleotide and a second fragment that is linear and free in solution; and c) detecting at least one of the cleavage products.

### BACKGROUND

During the past two decades, remarkable developments in molecular biology and genetics have produced a revolutionary growth in understanding of the implication of genes in human disease. Genes have been shown to be directly causative of certain disease states. For example, it has long been known that sickle cell anemia is caused by a single mutation in the human beta globin gene. In many other cases, genes play a role together with environmental factors and/or other genes to either cause disease or increase susceptibility to disease. Prominent examples of such conditions include the role of DNA sequence variation in ApoE in Alzheimer's disease, CKR5 in susceptibility to infection by HIV, Factor V in risk of deep venous thrombosis, MTHFR in cardiovascular disease and neural tube defects, p53 in HPV infection, various cytochrome p450s in drug metabolism, and HLA in autoimmune disease.

The genetic variations that lead to gene involvement in human disease are relatively small. Approximately 1% of the DNA bases which comprise the human genome contain polymorphisms that vary at least 1% of the time in the human population. The genomes of all organisms, including humans, undergo spontaneous mutation in the course of their continuing evolution. The majority of such mutations create polymorphisms, thus the mutated sequence and the initial sequence co-exist in the species population. However, the majority of DNA base differences are functionally inconsequential in that they affect neither the amino acid sequence of encoded proteins nor the expression levels of the encoded proteins. Some polymorphisms that lie within genes or their promoters do have a phenotypic effect and it is this small proportion of the genome's variation that accounts for the genetic component of all difference between individuals, e.g., physical appearance, disease susceptibility, disease resistance, and responsiveness to drug treatments.

One of the major forms of sequence variation in the human genome consists of single nucleotide polymorphisms (“SNPs”). Other forms of variation include copy number variations (CNVs) as well as short tandem repeats (including microsatellites), long tandem repeats (minisatellite), and other insertions and deletions. A SNP is a position (the “SNP site”, “SNP position” or “SNP nucleotide position”) at which at least two alternative bases occur, each of which at an appreciable frequency (i.e., >1%) in the human population. A SNP is said to be “allelic” in that due to the existence of the polymorphism, some members of a species may have the unmutated sequence (i.e., the original “allele”) whereas other members may have a mutated sequence (i.e., the variant or mutant allele). In the simplest case, only one mutated sequence may exist, and the polymorphism is said to be diallelic. The occurrence of alternative mutations can give rise to triallelic polymorphisms, etc. SNPs are widespread throughout the genome and SNPs that alter the function of a gene may be direct contributors to phenotypic variation. Due to their prevalence and widespread nature, SNPs are important diagnostic tools.

This disclosure relates to the detection of SNPs and other sequence variations.

### SUMMARY

A method of sample analysis is provided. In certain embodiments, the method may comprise: a) contacting a plurality of Q probes with a nucleic acid sample comprising a target polynucleotide under hybridization conditions to form a plurality of flap endonuclease substrates each comprising a Q probe and a site in the target polynucleotide; b) contacting the plurality of flap endonuclease substrates with a flap endonuclease under cleavage conditions to produce cleavage products, in which each of the Q probes of the flap endonuclease substrates is cleaved to produce cleavage products that include at least a first fragment that is hybridized with a site in the target polynucleotide and a second fragment that is linear and free in solution; and c) detecting at least one of the cleavage products.

### BRIEF DESCRIPTION OF THE FIGURES

FIG. 1 schematically illustrates a Q probe hybridized to a target polynucleotide.

FIG. 2 schematically illustrates certain features of one embodiment of a method that involves a Q probe and a target polynucleotide.

FIG. 3 schematically illustrates certain features of one embodiment of the method described herein.

FIG. 4 schematically illustrates certain features of another embodiment of the method described herein.

FIG. 5 schematically illustrates certain features of another embodiment of the method described herein.

FIG. 6 schematically illustrates certain features of another embodiment of the method described herein.

FIG. 7 shows electrophoresis data from a cleavage assay using Q probes.

FIG. 8 shows electrophoresis data from a ligation assay using Q probes.

FIG. 9 shows electrophoresis data from a rolling circle amplification assay using Q probes.

FIG. 10 shows electrophoresis data from a cleavage assay using fluorescently-labeled Q probes.

### DEFINITIONS

The term “sample” as used herein relates to a material or mixture of materials, typically, although not necessarily, in liquid form, containing one or more analytes of interest.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The term “nucleic acid sample,” as used herein denotes a sample containing nucleic acids.

The term “target polynucleotide,” as use herein, refers to a polynucleotide of interest under study. In certain embodiments, a target polynucleotide contains one or more target sites that are of interest under study.

The term “oligonucleotide” as used herein denotes a single stranded multimer of nucleotide of from about 2 to 200 nucleotides. Oligonucleotides may be synthetic or may be made enzymatically, and, in some embodiments, are 10 to 50 nucleotides in length. Oligonucleotides may contain ribonucleotide monomers (i.e., may be oligoribonucleotides) or deoxyribonucleotide monomers. An oligonucleotide may be 10 to 20, 11 to 30, 31 to 40, 41 to 50, 51-60, 61 to 70, 71 to 80, 80 to 100, 100 to 150 or 150 to 200 nucleotides in length. for example.

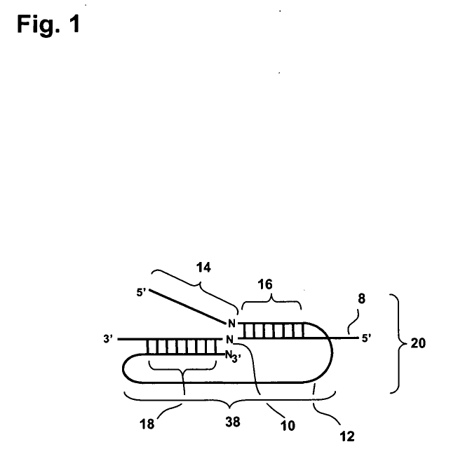

The term “duplex,” or “duplexed,” as used herein, describes two complementary polynucleotides that are base-paired, i.e., hybridized together. A duplex containing a Q probe and a target site is indicated as element 16 in the schematic illustration of FIG. 1.

The term “primer” as used herein refers to an oligonucleotide that has a nucleotide sequence that is complementary to a region of a target polynucleotide. A primer binds to the complementary region and is extended, using the target nucleic acid as the template, under primer extension conditions. A primer may be in the range of about 20 to about 60 nucleotides although primers outside of this length may be used.

The term “extending” as used herein refers to any addition of one or more nucleotides to the end of a nucleic acid, e.g. by ligation of an oligonucleotide or by using a polymerase.

The term “amplifying” as used herein refers to generating one or more copies of a target nucleic acid, using the target nucleic acid as a template.

An “array,” includes any two-dimensional or substantially two-dimensional (as well as a three-dimensional) arrangement of spatially addressable regions bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a surface of the substrate. Depending upon the use, any or all of the arrays may be the same or different from one another and each may contain multiple spots or features. An array may contain one or more, including more than two, more than ten, more than one hundred, more than one thousand, more ten thousand features, or even more than one hundred thousand features, in an area of less than 20 cm² or even less than 10 cm², e.g., less than about 5cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 mm and less than 1.5 mm, such as more than about 0.8 mm and less than about 1.2 mm.

Arrays can be fabricated using drop deposition from pulse-jets of either precursor units (such as nucleotide or amino acid monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different moieties (e.g., different oligonucleotide sequences) such that a region (i.e., a “feature” or “spot” of the array) at a particular predetermined location (i.e., an “address”) on the array contains a particular sequence. Array features are typically, but need not be, separated by intervening spaces.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

As used herein, the term “Tₘ” refers to the melting temperature an oligonucleotide duplex at which half of the duplexes remain hybridized and half of the duplexes dissociate into single strands. As Q probes contain more than one region which participate in a duplex, Tₘ of a Q probe refers to the melting temperature of the entire complex, i.e., the temperature at which half of the Q probes are completely dissociated from their target polynucleotides. The Tₘ of an oligonucleotide duplex may be experimentally determined or predicted using the following formula Tₘ=81.5+16.6(log₁₀[Na⁺])+0.41 (fraction G+C)−(60/N), where N is the chain length and [Na⁺] is less than 1 M. See Sambrook and Russell (2001; Molecular Cloning: A Laboratory Manual, 3^{rd} ed., Cold Spring Harbor Press, Cold Spring Harbor N.Y., ch. 10). Other formulas for predicting Tₘ of oligonucleotide duplexes exist and one formula may be more or less appropriate for a given condition or set of conditions.

As used herein, the term “Tₘ-matched” refers to a plurality of nucleic acid duplexes having Tₘs that are within a defined range.

The term “low-stringency hybridization conditions” as used herein refers to hybridization conditions that are suitable for hybridization of a flap oligonucleotide and a surface-tethered oligonucleotide that has a region that is complementary to the flap oligonucleotide. Such conditions may differ from one experiment to the next depending on the length and the nucleotide content of the complementary region. In certain cases, the temperature for low-stringency hybridization is 5°-10° C. lower than the calculated Tₘ of the resulting duplex under the conditions used.

As used herein “high-stringency wash conditions” refers to wash conditions that provide for disassociation of non-specifically bound oligonucleotides, but not disassociation of the desired hybridization target oligonucleotides containing the complementary sequence to the surface-tethered oligonucleotide. Such conditions release oligonucleotides that differ in sequence or length by one or more nucleotides from the desired hybridization target, but do not release the flap oligonucleotides from the surface-tethered oligonucleotide. Again, such conditions may differ from one experiment to the next depending on the length and the nucleotide content of the complementary region. In certain cases described in more detail below, the temperature for a high stringency wash may be 5°-10° C. lower than the calculated Tₘ of an extended duplex, and 5°-10° C. higher than the calculated Tₘ of a non-extended duplex, under the conditions used.

As used herein, the term “single nucleotide polymorphism”, or “SNP” for short, refers to single nucleotide position in a genomic sequence for which two or more alternative alleles are present at appreciable frequency (e.g., at least 1%) in a population.

As used herein, the term “SNP nucleotide” refers to a nucleotide that is the same as or complementary to a SNP. In certain embodiments, a SNP nucleotide is the terminal nucleotide in a flap oligonucleotide, and is used to identify the SNP in a target.

The term “flap endonuclease” or “FEN” for short, as used herein, refers a class of nucleolytic enzymes that act as structure specific endonucleases on DNA structures with a duplex containing a single stranded 5′ overhang, or flap, on one of the strands that is displaced by another strand of nucleic acid, i.e., such that there are overlapping nucleotides at the junction between the single and double-stranded DNA. FENs catalyze hydrolytic cleavage of the phosphodiester bond at the junction of single and double stranded DNA, releasing the overhang, or the flap. Flap endonucleases are reviewed by Ceska and Savers (Trends Biochem. Sci. 1998 23:331-336) and Liu et al (Annu. Rev. Biochem. 2004 73: 589-615). FENs may be individual enzymes, multi-subunit enzymes, or may exist as an activity of another enzyme or protein complex, e.g., a DNA polymerase.

The term “flap endonuclease substrate”, as used herein, refers to a nucleic acid complex that can be cleaved by a flap endonuclease to produce cleavage products.

The term “cleavage products”, as used herein, refers to products resulted from a flap endonuclease-mediated cleavage reaction on a flap endonuclease substrate.

As used herein, the term “flap oligonucleotide” or “flap” refers to a single-stranded oligonucleotide that is a cleavage product of a flap assay. When a Q probe is cleaved, the flap is located at the 5′ end of a Q probe and is not bound to the target polynucleotide.

As used herein, the term “overlap-dependent cleavage assay” or a “flap assay” refers to an assay in which a Q probe is cleaved by a flap endonuclease to release a flap oligonucleotide, where cleavage only occurs when there is an overlapping oligonucleotide at the junction between the single-stranded flap and the double-stranded DNA.

The term “Q probe,” as used herein, refers to an oligonucleotide, that, when bound to a target polynucleotide, forms a “Q”-shaped substrate for a flap endonuclease, where the tail of the Q (i.e., the flap oligonucleotide or the “first fragment”) is cleaved off in the cleavage reaction and the remainder of the Q probe is circular in shape (i.e., the “second fragment”) and remains bound to the target polynucleotide. At the time of cleavage, the ends of the second fragment base pair with nucleotides in the target polynucleotide that are immediately adjacent to each other. At the time of cleavage, the ends of the second fragment are not covalently linked. However, the ends can be ligated together after cleavage. A flap endonuclease substrate containing a Q probe bound to a target polynucleotide is schematically illustrated in FIG. 1. FIG. 1 shows flap endonuclease substrate 20 comprising Q probe 12 and a binding site in a target polynucleotide 8.

The Q probe of flap endonuclease substrate 20 contains a single-stranded flap region 14 and two regions 16 and 18 that are base-paired with the target polynucleotide in opposite directions. Regions 16 and 18 are considered the duplex regions in a flap endonuclease. Regions 16 and 18 are linked by a single-stranded segment 38 that loops from one end of the complex to the other. Nucleotide “N” is the overlapping nucleotide, where cleavage of flap endonuclease substrate 20 occurs immediately 3′ to the overlapping nucleotide that is adjacent to flap 14. In order of 5′to 3′, the Q probe of a flap endonuclease substrate containing a Q probe comprises: 1) a single stranded flap oligonucleotide 14, that contains a first overlap nucleotide that is complementary to the target nucleotide 10, 2) a first region 16 that is duplexed with a first segment of a binding site in the target polynucleotide, where the first region 16 binds to a nucleotide sequence that is 5′ to the target nucleotide 10, 3) a segment 38 that loops from one end of the flap endonuclease to substrate to the other, 4) a second region 18 that is duplexed with a second segment of the binding site in the target nucleotide, where second region 18 binds to a nucleotide sequence that is 3′ to target nucleotide 10 in an orientation that is opposite to that of the first region, and 5) a second overlap nucleotide that may be complementary to target nucleotide 10. The segment 38 that loops from one end of the substrate to the other may be single stranded or, in other embodiments, may contain double-stranded regions if a splint oligonucleotide is used, or if there are internal regions of complementarity in that segment. As shown in FIG. 1, the binding site for a Q probe contains a target nucleotide (which may be a site of sequence polymorphism, e.g., a SNP) flanked by sites to which the Q probe binds.

The term “free in solution,” as used here, describes a molecule, such as a polynucleotide, that is not bound or tethered to another molecule.

The term “denaturing,” as used herein, refers to the separation of a nucleic acid duplex into two single strands.

The term “intramolecularly ligated product,” as used herein, refers to a polynucleotide with its 5′ end and 3′ end ligated to each other, forming a covalently linked circular polynucleotide.

### DESCRIPTION OF EXEMPLARY EMBODIMENTS

A method of sample analysis is provided. In certain embodiments, the method may comprise: a) contacting a plurality of Q probes with a nucleic acid sample comprising a target polynucleotide under hybridization conditions to form a plurality of flap endonuclease substrates each comprising a Q probe and a site in the target polynucleotide; b) contacting the plurality of flap endonuclease substrates with a flap endonuclease under cleavage conditions to produce cleavage products, in which each of the Q probes of the flap endonuclease substrates is cleaved to produce cleavage products that include at least a first fragment that is hybridized with a site in the target polynucleotide and a second fragment that is linear and free in solution; and c) detecting at least one of the cleavage products.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Method for Sample Analysis

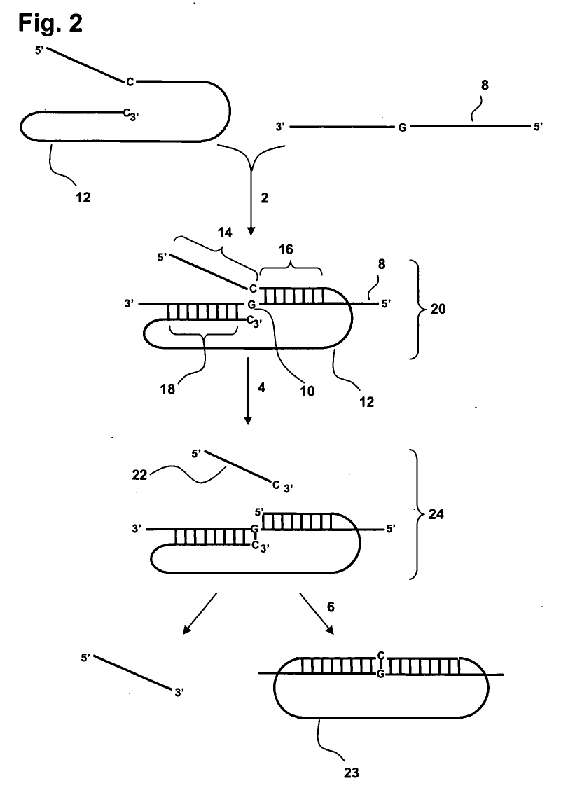

Certain features of the subject method are illustrated in FIG. 2 and are described in greater detail below. With reference to FIG. 2, the method includes contacting 2 a plurality of Q probes 12, with a nucleic acid sample comprising a target polynucleotide 8 under suitable hybridization conditions to form a plurality of flap endonuclease substrates 20. Each of the plurality of flap endonuclease substrates contains an overlapping nucleotide 10 that creates a specific structure recognizable by the flap endonuclease.

The flap endonuclease substrate produced by contacting contains a site in the target polynucleotide 8 and Q probe 12 containing the following sequence elements in the order of 5′ to 3′: 1) a single stranded flap oligonucleotide 14 containing a first overlap nucleotide that is complementary to the target nucleotide 10, 2) a first region 16 that is duplexed with a first segment of a binding site in the target polynucleotide, where the first region 16 binds to a nucleotide sequence that is 5′ to the target nucleotide 10, 3) a segment that loops from one end of the substrate to the other, 4) a second region 18 that is duplexed with a second segment of the binding site in the target nucleotide, where second region 18 binds to a nucleotide sequence that is 3′ to target nucleotide 10 in an orientation that is opposite to that of the first region, and 5) a second overlap nucleotide that may be complementary to target nucleotide 10.

As shown in FIG. 2, in the preferred embodiment the overlapping region recognizable by the flap endonuclease features complementarity of the target nucleotide 10 in the target polynucleotide to two nucleotides in the Q probe. One of the two nucleotides is the 3′ terminal nucleotide of the single-stranded flap 14. The other overlapping nucleotide is located at the 3′ end of the second region 18. In other embodiments, the overlapping nucleotide located at the 3′ end of the second region 18 may not be complementary to the target nucleotide 10. In still other embodiments, the overlapping region located at the 3′ end of the second region 18 may contain more than one nucleotide which is not complementary to the target nucleotide 10, e.g., two or more nucleotides. In these embodiments, the “double-flap” substrate may still be specifically cleaved by a flap endonuclease.

The contacting step 2 is done in the presence of a plurality of Q probes with a nucleic acid sample containing a target polynucleotide comprising binding sites for the plurality of Q probes. In certain cases, the method may be performed using at least 2, at least 4, at least 10, at least 100, at least 1,000, up to 5,000, at least 10,000 or at least 100,000 or more different Q probes in one assay.

In certain embodiments, many different flap endonuclease substrates may be formed as a result of the contacting step 2. In certain cases, the flap endonuclease substrates are Tₘ-matched within the plurality, such that they all denature and anneal within a certain temperature range, e.g., within about 20, 15, 10, 5, 2, or 1° C. of a chosen Tₘ.

After the contacting step 2, the flap endonuclease substrates are then contacted with a flap endonuclease under cleavage conditions 4 to result in cleavage products 24 derived from the Q probe 12. The cleavage products include at least a first fragment, such as flap oligonucleotide 22 in FIG. 2, and a second fragment, such as element 23 in FIG. 2. Either of the first fragment or the second fragment may be detected to determine whether flap endonuclease cleavage has occurred.

In certain cases, the method may involve multiple rounds of denaturing, reannealing, and cleaving in the same reaction vessel in order to provide more cleavage products.

A variety of methods may be used to identify which of the Q-probes are cleaved. For example, the fragments in the population of cleavage products may be sequenced, hybridized to an array, amplified by polymerase chain reaction using sequence-specific primers, or identified by size.

In certain embodiments, the flap oligonucleotides 22 produced by cleavage may be detected. In certain cases, the 3′ terminal nucleotide of the flap oligonucleotide is complementary to the target nucleotide 10 in the target polynucleotide. As such, determining which flap oligonucleotides have been cleaved identifies the target nucleotide of the sample polynucleotide.

In alternative embodiments, the second fragment produced by cleavage of the Q probe may be detected. As shown in FIG. 2, the second fragment may remain annealed to the target polynucleotide after contacting under cleavage conditions 4. The second fragment may be subjected to a ligation reaction 6 to create an intramolecular circular oligonucleotide 23. In certain cases, the circular oligonucleotide may be further amplified before detection. If this fragment 23 is pre-labeled with a purification tag, the fragment may then be purified and detected by separation in a gel or over a column. The second fragment may also be hybridized to an array to identify its sequence.

In certain cases, the hybridization and cleavage may be done at the same or at different temperatures. In certain embodiments, the hybridization and cleavage conditions may comprise a temperature that is at least 1, at least 5, at least 10, at least 15, or at least 20° C. or more higher than the calculated Tₘ of either duplex region (e.g., those duplexes formed by segments 16 and 18 in FIG. 1) formed by the Q probe and the site of the target polynucleotide. In certain cases, the hybridization and cleavage may be done at a temperature that is in the range of about 40° to 95°, e.g., 50° to 90°, or 60° to 80° C. For example, the condition may be at a temperature between 65° and 75° C.

The target polynucleotide 8 may be derived from a genomic source of any organism or virus. The organism may be a prokaryote or a eukaryote. In certain cases, the organism may be a plant or an animal, including reptiles, mammals, birds, fish, and amphibians. In other cases, the target polynucleotide is derived from the genomic source of a human or a rodent, such as a mouse or a rat. The genomic source often contains genomic DNA that may be purified or further enriched for a particular target polynucleotide. Methods of preparing genomic DNA for analysis is routine and known in the art, such as those described by Ausubel, F. M. et al., (Short protocols in molecular biology, 3rd ed., 1995, John Wiley & Sons, Inc., New York) and Sambrook, J. et al. (Molecular cloning: A laboratory manual, 2^{nd} ed., 1989, Cold Spring Harbor Laboratory Press, Cold Spring Harbor, N.Y.). In certain cases, the target polynucleotide may be a version of genomic DNA that is amplified, fragmented, or rearranged. In other cases, the target polynucleotide may be RNA or cDNA.

The subject method employs contacting a nucleic acid sample containing a target polynucleotide described above with a plurality of Q probes. As noted previously, Q probe is an oligonucleotide that when hybridized to a site in a target polynucleotide creates a complex with a structure recognizable by a flap endonuclease. In certain embodiments, each Q probe consists of one oligonucleotide of about 50 to 150, or about 100 to 200, e.g., 75 to 125, or 90 to 100 nucleotides in length.

A Q probe bound to a site in a target polynucleotide has a 5′ single-stranded flap and two double-stranded regions connected by a single stranded segment. The single-stranded flap (element 14) and the two double-stranded segments (elements 16 and 18) define the target specificity of the Q probe. The 5′ single-stranded flap may be about less than 50, 40, 30, or 20 nucleotides in length. For example, the flap may be about 20 nucleotides long. The single-stranded flap 14 contains a nucleotide at the 3′ end of the flap. If this 3′ terminal nucleotide and another nucleotide 3′ to the second double stranded segment 18 are complementary to the target nucleotide 10 of the sample polynucleotide, an overlapping structure recognizable by the flap endonuclease may be formed. In certain embodiments, the 3′ terminal nucleotide of the single-stranded flap is allele-specific. In certain cases, the 3′ terminal nucleotide is complementary to a site of single-nucleotide polymorphism (SNP).

As for the double-stranded segments of the Q probe, they may each be about 8 to 40, e.g., 10 to 15, 15 to 25 or 20 to 30 nucleotides in length. In certain cases, a substrate may comprise a double stranded region of up to 9, e.g., 6 to 9 or 8 to 9 nucleotides. These regions of the Q probe that participate in the formation of the first and second double-stranded segments (16 and 18) are sequence-specific, so to be optimized for binding to a specific location in the genomic DNA. These two double-stranded regions together in the context of the Q probe also define the Tₘ of the flap endonuclease substrates, such that in a single multiplex reaction vessel, all the flap endonuclease substrates may be Tₘ-matched.

The single-stranded segment 38 connecting the two double stranded segments may be about 25 to 90, e.g., 30 to 60, 40 to 50 nucleotides long. In certain cases, the length or sequence of this segment 38 may influence the Tₘ of the flap endonuclease substrates. This single-stranded segment may be of any linker sequence, or may be homopolymeric. In embodiments, this connecting segment may comprise primer sequences, unique barcode sequences, or other features which may be useful in downstream analysis. In particular embodiments, it may be useful to design this segment to minimize hybridization of the Q probe to non-target sequences. In certain embodiments, this connecting segment may comprise chemicals other than DNA nucleotides, such as RNA, peptides, carbohydrates, synthetic polymers such as polyethylene glycol, etc.

Since the nucleotide sequences of hundreds of thousand of SNPs from humans, other mammals (e.g., mice), and a variety of different plants (e.g., corn, rice and soybean), are known (see, e.g., Riva et al 2004, A SNP-centric database for the investigation of the human genome BMC Bioinformatics 5:33; McCarthy et al 2000 The use of single-nucleotide polymorphism maps in pharmacogenomics Nat Biotechnology 18:505-8) and are available in public databases (e.g., NCBI's online dbSNP database, and the online database of the International HapMap Project; see also Teufel et al 2006 Current bioinformatics tools in genomic biomedical research Int. J. Mol. Med. 17:967-73) the design of Q probes to be allele-specific or SNP-specific is well within the skill of one of skilled in the art. The SNP should be known prior to design of a set of Q probes. The SNP may be linked to a phenotype (e.g., a disease) or may be unlinked to a phenotype (e.g., may be an “anonymous” SNP).

In certain cases, the Q probes may be “Tₘ-matched” in that they are designed to have a similar melting temperature when complexed to their respective target polynucleotides (e.g., within about 20, 15, 10, 5, 2, or 1° C. of a chosen Tₘ) under the hybridization conditions used. The Tₘ of complex oligonucleotides may be calculated using conventional methods, e.g., in silico or experimentally. It will be recognized by one with skill in the art that the Q probe-target complex has two duplex regions that may form or melt independently.

For example, the Tₘ of Q probe-target complex may be a value higher than the Tₘ of either double-stranded region alone (e.g., those duplexes formed by segments 16 and 18 in FIG. 1), yet lower than a duplex DNA consisting of the same region of the target polynucleotide bound to its complementary sequence.

For certain Q probes, the Tₘ of the oligonucleotide duplex formed between the oligonucleotide and the matched or mismatched target for the polynucleotide in the genome under examination may be reduced by one or more destabilizing elements in the Q probe. Such elements include, but are not limited to, nucleotide substitutions and non-naturally occurring nucleotides that introduce a destabilizing mis-match between the Q probe and the target sequence, as well as insertions and deletions of nucleotides. Exemplary destabilizing elements are described in, for example, published U.S. patent application 2007008730, by Curry. A single Q probe may contain 1, 2, 3, 4, 5, 6, 7, 8, 9 or 10 or more destabilizing elements, depending on the length of the Q probe and the desired Tₘ. The destabilizing elements may be proximal to the 3′ terminal nucleotide of the flap, or distributed throughout the oligonucleotide. In certain cases, the destabilizing elements are distributed evenly throughout the Q probe. In particular embodiments, a subject Q probe may contain so-called unstructured nucleic acid nucleotides (UNAs), which nucleotides are known and may be synthesized synthetically (Kutyavin et al., Nucl. Acids Res. (2002) 30:4952-4959).

In other embodiments, the flap endonuclease substrate containing a Q probe and a site in the target polynucleotide may be destabilized by the use of destabilizing agents that are present in the hybridization buffer. Such elements include urea, and formamide, for example.

In certain embodiments, the Tₘ of a Q probe is pre-selected such that, in the hybridization conditions used, the Tₘ of a duplex containing a Q probe and its correctly matched target sequence (i.e., the target nucleotide in the polynucleotide) is higher than the hybridization temperature to be used, and the Tₘ of a complex containing a Q probe and its mismatched target sequence (i.e., the target nucleotide of the sample polynucleotide) is lower than the hybridization temperature to be used. For example, if the hybridization temperature is 65° C., then a Q probe may be designed such that the Tₘ of a complex containing that Q probe and its respective site in a target polynucleotide may be designed to be 66° C., and the complex containing that oligonucleotide and a mis-matched site may be designed to be 63° C.

In general, methods for the preparation of oligonucleotides are well known in the art (see, e.g., Harrington et al, Curr. Opin. Microbiol. (2000) 3:285-91, and Lipshutz et al., Nat. Genet. (1999) 21:20-4) and need not be described in any great detail. Molecular inversion probes or padlock probes are well-known in the art and their method of synthesis can also be adapted for the synthesis of Q probes. Disclosure of molecular inversion probes may be found in U.S. Pat. No. 7,074,564 and Pub No. 2005/0037356 and other references such as Absalan F, Ronaghi M. (Molecular inversion probe assay. Methods Mol Biol. 2007, 396:315-30), Nilsson M et al. (Padlock probes: circularizing oligonucleotides for localized DNA detection. Science. 1994, 265:2085-8).

In certain embodiments, Q probes are synthesized on a solid support in an array, where the oligonucleotides are grown in situ. Oligonucleotide arrays can be fabricated using any means, including drop deposition from pulse jets or from fluid-filled tips, etc, or using photolithographic means. Polynucleotide precursor units (such as nucleotide monomers), in the case of in situ fabrication can be deposited. Oligonucleotides synthesized on a solid support may then be cleaved off to generate a library of oligonucleotides. Such methods are described in detail in, for example U.S. Pat. Nos. 7,385,050, 6,222,030, 6,323,043, and US Pat Pub No. 2002/0058802, etc., the disclosures of which are herein incorporated by reference. The oligos may be tethered to a solid support via a cleavable linker, and cleaved from the support before use.

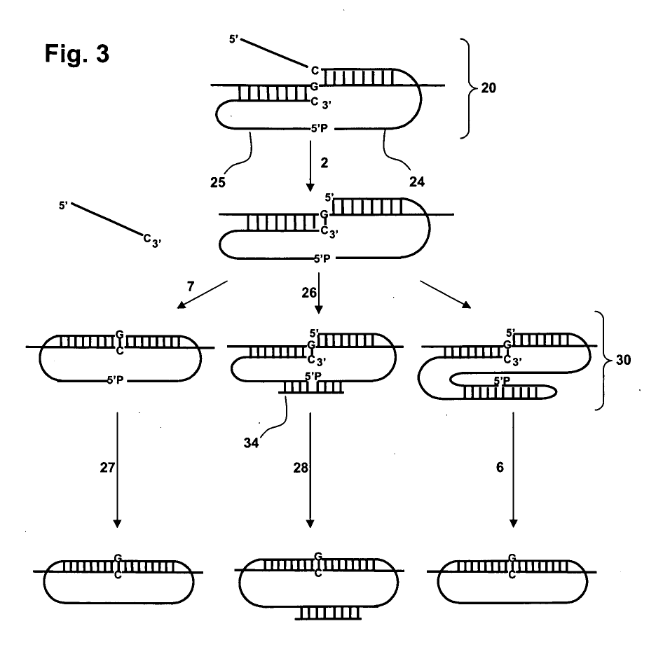

In alternative embodiments, a Q probe may contain two oligonucleotides (2-oligo Q probe), such that when the two oligonucleotides are hybridized to a site in the target polynucleotide, a complex is formed with the same structure as when the Q probe is of one oligonucleotide. This embodiment is shown in FIG. 3. The difference is that the single-stranded segment connecting the two double-stranded segments is not contiguous. An example of a Q probe containing of two oligonucleotides is illustrated in FIG. 3 as elements 24 and 25. When a 2-oligo Q probe hybridizes to a site in the target polynucleotide, a flap endonuclease substrate is formed. Similar to FIG. 1, the flap endonuclease substrate comprises in the order of 5′ to 3′, 1) a first oligonucleotide comprising a 5′ single-stranded flap, a first double-stranded segment between the first oligonucleotide and a segment of the polynucleotide 5′ to the target nucleotide, and a 3′ single-stranded segment (referred herein as a first single-stranded segment) and, 2) a second oligonucleotide comprising a 5′ single-stranded segment (referred herein as a second single-stranded segment) and a second double-stranded segment between the second oligonucleotide and a segment of the polynucleotide 3′ to the target nucleotide.

In certain cases, the cleaved flap endonuclease substrate comprising a 2-oligo Q probe is ligated into a circular fragment by using a circligase, which ligates the two ends of the two oligonucleotides, as shown as step 27 in FIG. 3.

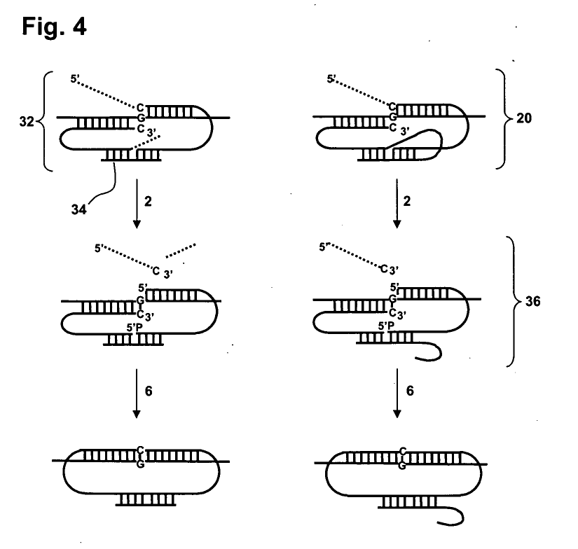

In certain embodiments, the structure of the flap endonuclease substrate comprising of a 2-oligo Q probe may be maintained with another oligonucleotide that serves as a splint, as shown as element 34 in FIGS. 3 and 4. The splint comprises a sequence complementary to at least a part of the first single-stranded segment of the first oligonucleotide and another sequence complementary to at least a part of the second single-stranded segment of the second oligonucleotide. Hybridization of the splint template to parts of the first and second oligonucleotides creates a third double stranded region. In certain embodiments, it may be advantageous to design the sequence of this third double stranded region to have a Tₘ higher than, equal to, or lower than the Tₘ of the Q probe-target complex. In certain cases, the cleaved flap endonuclease substrate comprising a 2-oligo Q probe hybridized to the splint is ligated into a circular fragment by a ligase, as shown as step 28 in FIG. 3.

In certain cases, this third double stranded region comprises a 5′ single-stranded flap contiguous to the end of the second single-stranded segment. This 5′ single-stranded flap contiguous to the end of the second single-stranded segment may be cleaved under cleavage conditions. In other words, the hybridization of the splint template to the Q probe may result in a complex with a second single-stranded flap at the 5′ end of the second oligonucleotide, in addition to the 5′ single-stranded flap of the first oligonucleotide. A complex with two single-stranded flaps is shown as element 32 in FIG. 4.

This complex may then be subjected to cleavage in two sites by a flap endonuclease to yield two single-stranded flap oligonucleotides. After cleavage, the first and second oligonucleotides may be ligated together to form a circular oligonucleotide like element 23 shown in FIG. 2.

In an alternative embodiment, the splint template is contiguous to an end of either the first single-stranded segment or the second single-stranded segment, in which the hybridization of the splint template to the first single-stranded segment and the second single-stranded segment produces a hairpin with a loop and a stem. The splint template forms a stem region of the hairpin loop. In certain cases, the splint template is a sequence within the 5′ single-stranded segment of the second oligonucleotide, such that a hairpin structure is formed, shown as elements 30 and 34 in FIGS. 3 and 4.

Depending on which sequences comprise of the stem-region of the hairpin, an additional cleavable single-stranded flap may or may not be formed. If the first single-stranded segment hybridizes to the region of the second oligonucleotide that is further away from the loop of the hairpin than the self-hybridization region of the second oligonucleotide, such as complex 30 in FIG. 3, no cleavable single-stranded flap from the second oligonucleotide is formed. Ligation of hairpin in complex 30 yields a circular oligonucleotide. In a different embodiment, if part of the first oligonucleotide hybridizes to the region of the second oligonucleotide that is closer to the loop of the hairpin than the self-hybridization region of the second oligonucleotide, such as complex 34 in FIG. 4, a cleavable single-stranded segment is formed as the loop of the hairpin. Complex 34 may then be subjected to cleavage in two sites by a flap endonuclease to yield a 5′ single-stranded segment from the first oligonucleotide and third double-stranded region comprising a splint template with an overhang.

As discussed previously, the plurality of Q probes employed in the subject method when hybridized to their respective targets forms complexes that are Tₘ-matched. In certain embodiments, the concentration of Q probes for each respective target may be less than about 10,000×, 1000×, or 100× molar excess. In certain cases, the Q probes are labeled. The labeled may be linked to the 5′ single-stranded flap and/or other parts of the Q probe. For each polynucleotide containing a specific target nucleotide, there may be one type of Q probe. In other cases, for each polynucleotide containing a specific target nucleotide, there may be a pair of Q probe, each designed to hybridize to an allelic variant. In certain cases, a Q probe within the same pair may be labeled differently, such that a unique tag or dye identifies one allelic variant. For example, a pair of Q probe may be designed to detect a cytosine or an adenine as the target nucleotide in the sample polynucleotide. For example, a Q probe containing guanine at the 3′ terminal of the flap may be labeled green, while the other Q probe of the pair containing thymine at the 3′ terminal of the flap may be labeled red.

The cleavage step of the subject method employs enzymes having flap endonuclease activity. The flap endonucleases may be of a eukaryotic, a prokaryotic, an archaea, or of a viral origin. In certain cases, FEN enzyme may be a Taq polymerase, flap endonuclease I, an N-terminal domain of DNA polymerase I or thermostable variants thereof. As noted above, the subject method comprises of contacting a plurality of Q probes with a nucleic acid sample comprising a target nucleotide under hybridization conditions to form a plurality of flap endonuclease substrates, contacting the substrates with a flap endonuclease, and detecting the cleavage products. The cleavage structure recognizable by the flap endonuclease is formed by overlapping nucleotides at the target nucleotide, as explained above.

As such, a successful cleavage reaction indicates complementarity of the nucleotides. Since the sequence of the Q probe is known beforehand, detection of cleavage products indicates that the target specific for the Q probe exists in the sample. In certain cases, detection of the cleavage products serves as a positive detection of a particular allelic variant of the target polynucleotide under study.

In certain cases, the presence of a flap oligonucleotide may indicate the presence of a specific SNP. In such a cleavage reaction, a flap endonuclease activity (provided by FEN1 or other suitable enzymes) cleaves to produce a flap from a Q probe only when a complex is formed with specific complementary regions between nucleic acids, in which these complementary regions comprises the SNP. If a particular SNP is absent, no complementary regions would be present in the complex and no flap would be produced. Similar assays, which may be also known as INVADER® assays, are generally known in the art and are described in detail in Mast et al. (Mast et al. “INVADER® Assay for Single-Nucleotide Polymorphism Genotyping and Gene Copy Number Evaluation.” Methods in Mol. Biol. (2006) 335:173-186), and Stevens et al. (Stevens et al. “Analysis of single nucleotide polymorphisms with solid phase invasive cleavage reactions.” Nucleic Acids Res. (2001) 29:e77). Certain aspects of the flap endonuclease assay are also disclosed in U.S. Pat. No. 5,846,717, US Pat Pub No. 2006/0240419 and 2007/0003942.

In certain embodiments after the cleavage reaction, the sample containing the flap endonuclease substrates along with cleavage products are subjected to denaturation. The denaturing conditions separate the fragment annealed to the target polynucleotides. In certain cases, the cleavage fragments may be purified from the target polynucleotide. After denaturation, the sample containing the target polynucleotide may again contacted with a plurality of Q probes to produce additional flap endonuclease substrates, which is then subjected to cleavage conditions. The cycle of contacting the sample with Q probes and then contacting with a flap endonuclease may be iterated with denaturation in between cycles. This repetition may increase the number of cleavage products to be detected without changing the amount or concentration of the target polynucleotide in a nucleic acid sample.

A variety of methods may be used to detect a successful cleavage reaction, including running the reaction mixture in a gel or over a column and hybridization of the reaction to an array.

In certain embodiments, detection of the cleavage products comprises further analysis beyond detecting the mere presence of cleavage products. In a cleavage reaction with several different target nucleotides present in the same sample, a plurality of Q probes may be employed, comprising one type or one pair of Q probes for each target site. Hence, differentiation of the Q probes within the plurality may be performed in addition to detection the presence of cleavage products. In such a multiplex reaction, cleavage products derived from different Q probes may be differentiated within the plurality by several methods. Tagging the flap or other regions of the Q probe may aid in enriching for certain cleavage products. Array hybridization or parallel sequencing the plurality of cleavage products then allows identification of the cleavage product. Sequences of the flaps or other regions of the Q probe may serve as barcodes to identify the Q probes that have been cleaved.

In alternative embodiments, varying length of the single-stranded flap may be used to differentiate one Q probe from another. In another embodiment, varying the length of the circular oligonucleotide formed by ligation after cleavage reaction may also aid in differentiation within the plurality. The circular oligonucleotide may be also be enriched by degrading linear nucleic acids in the sample after ligation of the circular oligonucleotide. Separation of the cleavage products by size then identifies the type of Q probe that has been cleaved.

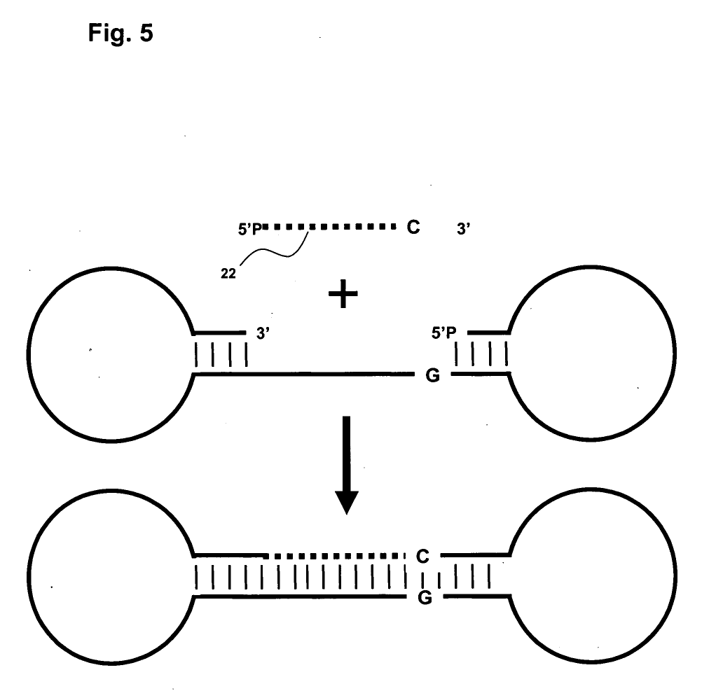

In certain embodiments, the single-stranded flap 22 and/or the ligated circular fragment may be amplified prior to detection. If the single-stranded flap is to be amplified, an embodiment may use rolling circle amplification pods in addition to the PCR method. An example of a rolling circle amplification pod is shown in FIG. 5. A rolling circle amplification pod polynucleotide may comprise, in the order of 5′ to 3′: 1) a 5′ phosphorylated end, 2) a double stranded region between the 5′ segment and the middle of the polynucleotide, 3) a single stranded loop region, 4) a double stranded region, 5) a single stranded region, which is complementary to the single stranded flap which is to be amplified, 6) a double stranded region, 7) a single stranded loop region, and 8) a a double stranded region between the 3′ segment and the middle of the polynucleotide. Hybridization of the single-stranded flap 22 in a sequence-specific manner to a rolling circle amplification pod enables the ligation of the pod to become a contiguous, circular polynucleotide. Once the polynucleotide is ligated to be contiguous, it can be primed and amplified. Successful amplification of the rolling circle amplification pod is another way to determine the sequence of the flap in addition to positively identifying a successful cleavage of the Q probe. The loop regions of the amplification pod may contain sequences which are complementary to primers used for amplification, and may also contain unique sequences such that each amplification pod may be identified. Combinations of primer sequences which are common to all amplification pods, or unique to individaul amplification pods, may be used to amplify pools of amplification pods or enrich for individual pods.

Ligation of the 3′ end of the flap oligonucleotide and the 5′ end of the amplification pod oligonucleotide requires a 5′ phosphate on the amplification pod. Furthermore, ligation of the 5′ end of the flap oligonucleotide and the 3′ end of the amplification pod oligonucleotide requires a 5′ phosphate on the flap. Therefore, the Q probes and amplification pods may be synthesized with a 5′ phosphate to enable the ligation of the cleavage products.

If the ligated circular fragment is to be amplified by polymerase chain reaction, the sequences of the various Q probe may be designed to be different such that PCR primers may anneal in different locations to create amplified products of different lengths. The circular fragment may also be amplified by rolling circle amplification.

In certain embodiments, the amplified products may further be analyzed by parallel sequencing or hybridization to an addressable array. The amplified product may hybridize to array probes to yield a positive signal. Linking the location of the probe on an addressable array to a database may provide the nucleotide sequence information of the amplified product hybridized to the probe. Using the sequence information, the identify of the Q probe and the target nucleotide in the sample polynucleotide may be deciphered.

In certain embodiments, one or more cleavage products may be circularized by intramolecular ligation. When the Q probe consists of only one oligonucleotide as shown in FIGS. 1 and 2, a ligase may be used to ligate the 5′ end and the 3′ end of the fragment of the Q probe. The resulted circular fragment may be subjected to amplification and detection as described above. If the Q probe employed consists of two oligonucleotides as shown in FIGS. 3 and 4, there are several embodiments of the subject method to circularize the cleavage products. Ligation of the ends overlapping the region of the target nucleotide may be done by a ligase. However, ligation of the 3′ end of the first oligonucleotide and the 5′ end of the second oligonucleotide, shown as elements 24 and 25, respectively, requires a 5′ phosphate. The Q probes may be synthesized with a 5′ phosphate on the second oligonucleotide to enable the ligation of the cleavage products. FIG. 3 illustrates several embodiments where ligation employs either a circligase or a splint template provided by another oligonucleotide or a sequence within the second oligonucleotide, as described previously.

In other cases where hybridization of the 2-oligo Q probes to a site in the target polynucleotide forms a complex with two cleavages sites, such as complexes 32 and 34, there is no need to synthesize a second oligonucleotide specifically with a 5′ phosphate. As shown in FIG. 4, the flap endonuclease cleavage reaction occurring at the region of the third double-stranded region leaves a 5′ phosphate available for subsequence ligation.

In certain embodiments and with reference to FIGS. 3 and 4, the method may involve: a) contacting a Q probe to a target site of a plurality of target polynucleotides to form flap endonuclease substrates, wherein the flap endonuclease substrate comprise: i) binding sites in the target polynucleotide, and ii) the Q probe, wherein the Q probe comprises, in order from 5′ to 3′: 1) a single-stranded flap, 2) a first double-stranded region between a segment of the Q probe and the binding sites 5′ to the target site, 3) a single-stranded segment, and 4) a second double-stranded region between a segment of the Q probe and binding sites 3′ to the target site.

In certain embodiments, the contacting step may comprise contacting a first oligonucleotide and a second oligonucleotide to each of the plurality of target polynucleotides containing a target site to form a flap endonuclease substrate containing i) binding sites in the target polynucleotides, ii) the first oligonucleotide, and iii) the second oligonucleotide, wherein the first oligonucleotide comprises, in order from 5′ to 3′: 1) a single-stranded flap, 2) a first double-stranded region between a segment of the first oligonucleotide and the binding sites 5′ to the target site, and 3) a first single-stranded segment, where the second oligonucleotide comprises, in order from 3′ to 5′: 1) a second double-stranded region between a segment of the second oligonucleotide and the binding sites 3′ to the target site, and 2) a second single-stranded segment. This method may involve ligating the first and the second oligonucleotide after the contacting step to produce a circular polynucleotide and in certain cases may employ a splint template that hybridizes to ends of the first single-stranded segment and of the second single-stranded segment to form a third double-stranded region. In particular embodiments, the ligating may employ a circligase.

### Method for Detecting Flap Oligonucleotides

In certain cases the subject method produces a plurality of flap oligonucleotide cleavage products that are in solution. By identifying which cleavage products are produced, the identity of the target nucleotide (e.g., an SNP) can be determined. Furthermore, the detection method can include steps which are dependent on the sequence of the 3′ terminal nucleotide of the flap (comprising the target nucleotide or its complement), conferring an additional level of specificity to the assay.

In one embodiment described in greater detail below, the detection method may include contacting a surface-tethered oligonucleotide with a sample comprising a flap oligonucleotide under hybridization conditions to provide for the hybridization of the flap oligonucleotide and the surface-tethered oligonucleotide. The method further includes extending the flap oligonucleotide to produce an extended duplex, subjecting the extended duplex to conditions that provide for its separation from the non-extended duplex (e.g. washing), and detecting the extended duplex. Such methods are described in U.S. patent application Ser. No. 12/013,378, filed on Jan. 11, 2008, which is incorporated herein by reference for disclosure of those methods.

This detection method generally includes contacting a surface-tethered oligonucleotide with a sample containing a plurality of flap oligonucleotides each having a different sequence under hybridization conditions to provide an overhang duplex. The duplexes are then extended using the overhang of the surface-tethered oligonucleotide as a template. If there is sufficient complementary between the surface-tethered oligonucleotide and the flap oligonucleotide, the flap oligonucleotide is extended to increase stability and Tₘ of a duplex. If there is insufficient complementary between the surface-tethered oligonucleotide and the flap oligonucleotide, the flap oligonucleotide is not extended and there is no change to the Tₘ of a duplex. The duplexes are then subjected to wash conditions that provide for disassociation of the non-extended duplexes but not the extended duplexes. Flap oligonucleotides that are extended can then be detected.

In certain embodiments, the contacting may produce an oligonucleotide duplex comprising a double-stranded surface-proximal region and a single-stranded surface-distal overhang. A single-stranded overhang is made up of additional nucleotides on the surface-tethered oligonucleotide beyond the region that is complementary to the flap oligonucleotide.

The contacting step of the method is generally performed under conditions suitable for annealing of a flap oligonucleotide to a surface-tethered oligonucleotide to produce an oligonucleotide duplex. As noted above, while such hybridization conditions may vary depending on the length and composition of the region of complementarity between the two oligonucleotides, suitable conditions are nevertheless known and described in, e.g., Sambrook et al, supra. In certain cases, conditions suitable for successful hybridization of a flap oligonucleotide and a surface-tethered oligonucleotide may be determined by calculating the Tₘ of the expected oligonucleotide duplex in a particular hybridization buffer using the formula Tₘ=81.5+16.6(log₁₀[Na⁺])+0.41 (fraction G+C)−(60/N), where N is the chain length and [Na⁺] is less than 1 M. In these cases, the hybridization temperature may be 2°-10° C., e.g., 5°-10° C., lower than the calculated Tₘ of the expected oligonucleotide duplex. Suitable hybridization conditions may also be determined experimentally.

After an oligonucleotide duplex is formed between surface-tethered oligonucleotide and flap oligonucleotide, the duplex is subjected to a template-dependent extension using the overhang as the template. In certain embodiments, a polymerase may be employed to add nucleotides, e.g., labeled nucleotides to the 3′ end of the flap oligonucleotide. In other cases, a ligase may be used to ligate an oligonucleotide, e.g. labeled oligonucleotides to an end of the flap oligonucleotide. By extending the oligonucleotide duplex, the length of the double-stranded region is increased. Consequently, the Tₘ of extended duplex is higher than the Tₘ of the duplex before extension or non-extended duplex. Further, the extension may also incorporate a label into the oligonucleotide duplex for subsequent detection.

In certain cases, the oligonucleotide duplex may contain regions that are not complementary, and, as such, may not be extended despite being subjected to extension conditions. The non-extended duplex may be, for example, a duplex comprising a surface-tethered oligonucleotide and a flap oligonucleotide that are not fully complementary, or a duplex in which the overhang-adjacent nucleotide is not complementary to the corresponding nucleotide in the surface-tethered nucleotide. For example, if a sample contains a flap oligonucleotide that is not perfectly matched to surface-proximal region of the surface-tethered oligonucleotide, such an oligonucleotide duplex formed by imperfectly matched oligonucleotides may not be extended. In another example, some flap oligonucleotides may include nucleotides beyond overhang-adjacent nucleotide which, if they are not complementary to the overhang, may not be extended. In a particular example, flap oligonucleotides which are part of uncleaved Q probes may not be extended.

After extension, the duplex is subjected to wash conditions that separate non-extended flap oligonucleotides, but not extended flap oligonucleotides, from the surface-tethered oligonucleotide. In certain cases, the wash comprises conditions that preferentially allow separation of the unextended oligonucleotide duplex as compared to the extended duplex. Since extension of the flap oligonucleotide exclusively increases the Tₘ of duplexes in which the flap oligonucleotides are extended, extended flap oligonucleotides and non-extended flap oligonucleotides can be discriminated. Only duplexes that have extended flap oligonucleotides will remain intact after washing and are detected by detecting the incorporated label. Since the Tₘ is increased for extended duplex compared to the Tₘ of non-extended duplex, the wash conditions are at a stringency that is higher than the hybridization conditions used. In certain embodiments, the temperature of the wash may be chosen so that it is 2°-10° C., e.g., 5°-10° C. lower than the Tₘ of an extended duplex but 2°-10° C., e.g., 5°-10° C. higher than the Tₘ of the non-extended duplex, under the conditions used. As would be recognized by one of skilled in the art, in certain cases the wash temperature may be higher than the hybridization temperature, e.g., by at least 5° C., at least 10° C. or at least 20° C., up to about 30° C. In other cases, the concentration of ions, e.g., Na⁺ in the wash buffer may be less than the concentration of ions in the hybridization buffer, e.g., by at least 50%, at least 80%, at least 90% or up to about 95%. In other embodiments, the wash may be done in a buffer containing less ions and at a lower temperature than the hybridization. Such conditions are readily calculable using the following formula: Tₘ=81.5+16.6(log₁₀[Na⁺])+0.41 (fraction G+C)−(60/N), where N is the chain length and [Na⁺] is less than 1 M, where the wash and hybridization temperatures are 2°-10° C. lower than the calculated Tₘ for an extended duplex and non-extended duplex, respectively. In other embodiments, the stringency of the wash buffer may be altered by changing the concentration of a denaturant such as formamide. Such hybridization and wash conditions and reagents, e.g., SSC, SSPE, etc., for making the same are described in great detail in Sambrook, supra.

The higher stringency of the wash conditions effectively separates non-extended duplexes from the flap oligonucleotide in the non-extended duplexes. The extended duplexes do not disassociate. The selective disassociation of non-extended duplexes allows for detection of extended flap oligonucleotides that are annealed to the surface-tethered oligonucleotides.

After subjecting the extended duplex to high-stringency wash conditions, the retained extended duplex may be detected by detecting a label, e.g. a fluorescent or a hapten label in the extended flap oligonucleotide. In certain embodiments, the label may already be present in a pre-labeled flap oligonucleotide or be incorporated during extension. For example, contacting an oligonucleotide duplex with a reagent mix containing polymerase and labeled nucleotides produces extended a duplex that is labeled. In certain embodiments, reagent mix comprises nucleotides of more than one type, in which one of the types of the nucleotides may be labeled and the other types are unlabeled. In these embodiments, the types of labeled and unlabeled nucleotides in the reagent mix could be chosen to control the number or type of labels added. In an embodiment, unlabeled nucleotides may extend the flap oligonucleotide and the labeled nucleotide is added as the terminal nucleotide. For example, an overhang of an oligonucleotide duplex may comprise of a stretch of cytosines followed by a thymine as the terminal nucleotide. In this example, an extension reaction would comprise of unlabeled guanines to complement the stretch of cytosines and labeled adenosines to complement the terminal nucleotide. In another example, modified labeled nucleotides such as dideoxynucleotides could be used to ensure the addition of a single label per oligonucleotide duplex.

In other words, in certain cases which Q probes that are cleaved can be determined using the following method: contacting a surface-tethered oligonucleotide with a sample comprising a flap oligonucleotide to produce an oligonucleotide duplex comprising a double-stranded surface-proximal region and a single-stranded surface-distal overhang; extending the flap oligonucleotide using the overhang as a template to produce an extended duplex; subjecting the extended duplex to a wash that separates the oligonucleotide duplex but does not separate the extended duplex; and detecting the extended duplex. The method may use wash conditions that preferentially separate the oligonucleotide duplex as compared to the extended duplex.

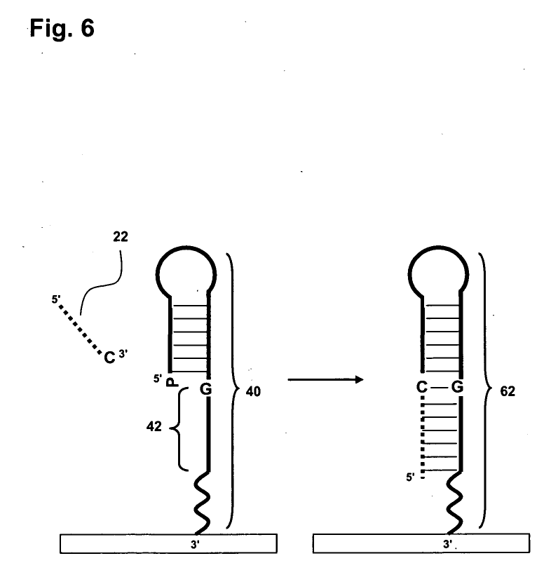

In an alternative embodiment shown in FIG. 6, the flap oligonucleotide 22 produced by cleavage of the Q probe can be ligated to a surface-tethered oligonucleotide, shown as element 40. In this embodiment, the surface-tethered oligonucleotide may contain a single-stranded surface-proximal region and a double-stranded surface-distal region comprising a sequence which can form a hairpin. The single-stranded surface-proximal region contains a region 42 which is complementary to the flap oligonucleotide. The surface-tethered oligonucleotide should contain a 5′ phosphate to enable ligation. When the flap oligonucleotide anneals to the surface-proximal region of the surface-tethered oligonucleotide, the flap oligonucleotide may be ligated to the surface-tethered oligonucleotide 40 to produce the longer surface-tethered oligonucleotide 62. If there is a mismatch between the 3′ end of the flap oligonucleotide 22 and the corresponding nucleotide in the surface-tethered oligonucleotide, ligation will be inhibited, and the flap oligonucleotide containing the mismatch can be removed in a high stringency wash. Furthermore, if there are additional nucleotides in the flap oligonucleotide which are 3′ to the target nucleotide, e.g., such as would be present in the uncleaved Q probe, ligation of the flap to the surface-tethered oligonucleotide will be inhibited. In this fashion, ligation to the surface-tethered oligonucleotide may confer additional specificity to the assay. After the correct flap oligonucleotides are ligated to their correct surface-tethered oligonucleotides, a wash or denaturation step may be performed to remove the unligated nucleic acids from the surface-tethered oligonucleotides. As the flap oligonucleotides will be covalently linked to the surface after ligation, a denaturing wash (e.g., addition of distilled water at 95° C.) may be applied without removing the ligated flap oligonucleotide. As would be recognized by one of skilled in the art, there are many denaturing conditions which would be sufficient to remove unligated nucleic acids while the ligated flap oligonucleotides remain tethered to the surface. If the flap oligonucleotide contains a label, the ligated flap oligonucleotides could be easily detected.

In other words, in certain cases which Q probes that are cleaved can be determined using the following method: contacting a surface-tethered oligonucleotide which contains a hairpin and a 5′ phosphate with a sample comprising a flap oligonucleotide to produce an oligonucleotide duplex; ligating the flap oligonucleotide to the surface-tethered oligonucleotide to produce a ligated duplex; subjecting the ligated duplex to a denaturing wash that separates the unligated nucleic acid duplexes but does not remove the ligated flap oligonucleotide; and detecting the ligated flap oligonucleotide.

### Compositions

A composition useful in the subject method is also provided. The subject composition comprises a plurality of Q probes, as described above, in which the hybridization of the Q probes to a plurality of target polynucleotides forms a plurality of flap endonuclease substrates. Each Q probe within the plurality may be designed to have a similar melting temperature when complexed to their respective target polynucleotides (e.g., within about 20, 15, 10, 5, 2, or 1° C. of a chosen Tₘ).

The Q probes in the subject composition may be synthesized by a variety of method as described above. In certain embodiments, the subject composition comprises of a plurality of Q probes in solution. In certain cases, the subject composition comprises of a plurality of Q probes tethered to a solid support in an array via a cleavable linker. In particular embodiments, the nucleotide sequence of the flap oligonucleotide region of the Q probes may be different from one another, allowing the identification of cleaved Q probes by hybridization.

### Kits

Also provided by the subject invention are kits for practicing the subject method, as described above. The subject kit contains a set of at least 10, at least 1,000, or at least 10,000 or more sequence-specific Q probes that when hybridized to a plurality of target polynucleotides forms a plurality of flap endonuclease substrates. In certain cases, the flap endonuclease substrates in the plurality are Tₘ-matched. In certain kits, the each type of Q probe in a plurality may be specific to only an allelic variant of the sequence under study. In certain kits, there is a pair of Q probes for each allelic variant of sequence under study. The kit may further contain a flap endonuclease and a reference sample to be employed in the subject method.

In additional embodiments, the kit further comprises an array of probes that are complementary to sequences of cleavage products. The kit may provide additional probe features on the array for positive and negative controls, depending on the analysis. The kit may also comprise a polymerase or tools for amplifying and purifying cleavage products, such as ligase, circligase, primers, and additional polynucleotides to serve as amplification pods.

The kits may be identified by the type of Q probes included and the chromosomal regions the Q probes are predicted to bind to. The kits may be further identified by the method of analyzing the cleavage products.

In addition to above-mentioned components, the subject kit typically further includes instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

In addition to the instructions, the kits may also include one or more control analyte mixtures, e.g., two or more control analytes for use in testing the kit.

### Utility

The subject method finds use in a variety of applications, where such applications generally include genomic DNA analysis applications in which the presence of a particular sequence polymorphism in a given sample is detected. In general, the method involves contacting a plurality of Q probes to a target polynucleotide to form flap endonuclease substrates, and contacting the flap endonuclease substrates to a flap endonuclease. After cleavage, cleavage products may be detected and analyzed.

The above-described method may be employed to analyze SNPs. In general terms, certain embodiments of the method may comprise: a) contacting a plurality of Q probes with a nucleic acid sample comprising a target polynucleotide containing a single nucleotide polymorphism for forming a plurality of flap endonuclease substrates each comprising a Q probe and a site in the target polynucleotide; b) contacting the plurality of flap endonuclease substrates with a flap endonuclease to produce cleavage products.

The target nucleotide of the polynucleotide may be the site of the SNP under study and the 3′ terminal nucleotide of the flap of the Q probe is specific to allelic variants of the SNP. Certain regions of the Q probe are complementary to sequences 5′ and 3′ to the SNP such that two double-stranded segments flanking the SNP are formed in the flap endonuclease substrates. Complementarity at the site of SNP creates an overlapping structure of nucleotides recognizable for cleavage by the flap endonuclease.

The subject method may be useful for the detection of different SNPs and at different sites of the genomic DNA by using a plurality of Q probes. When the Q probes are hybridized to their respective sites of target polynucleotides, Tₘ-matched flap endonuclease substrates are formed. Tₘ-matching allows the assay to be done in the same reaction vessel as hybridization and cleavage conditions may be done in the same temperature range. As such, the subject method and composition find use in a multiplex reaction assay.

Cleavage products from the plurality of Q probes may be identified by a variety of methods described above. Briefly, the length of the cleavage products, the sequence of the cleavage products, the amplification methods, differential labeling of Q probes corresponding to each allelic variant, etc., may enable identification of each cleavage product with its respective Q probe. In certain cases, the flexibility of using a Q probe consisting of one or two oligonucleotides permits the use and synthesis of Q probes that are different in a broad range of length and sequence specificity. The detection step is also not limited to detecting only one flap for each Q probe cleaved but multiple flaps or circularized fragments are also available for detection.

In certain embodiments, the specificity of Q probes and the multiplex feature of the subject method also allow a small ratio of molar concentration of Q probes to the concentration of the target polynucleotide. For example, the concentration of Q probes for each respective target may be less than about 10,000×, 1000×, or 100× molar excess. In effect, each assay may be cost-efficient, while minimizing background signals. In addition, the hybridization and cleavage conditions may also be tailored for high temperatures due to high stability of the flap endonuclease substrates, further increasing specificity in a multiplex reaction.

The subject method finds use in a variety of diagnostic and research purposes since nucleotide polymorphism plays an important role in conditions relevant to human diseases and genomic evolution of many organisms.

In particular, the above-described methods may be employed to diagnose, predict or investigate cancerous condition or other mammalian diseases, including but not limited to, leukemia, breast carcinoma, prostate cancer, Alzheimer's disease, Parkinsons's disease, epilepsy, amylotrophic lateral schlerosis, multiple sclerosis, stroke, autism, mental retardation, and developmental disorders. Many nucleotide polymorphisms are associated with and are thought to be a factor in producing these disorders. Knowing the type and the location of the nucleotide polymorphism may greatly aid the diagnosis, prognosis, and understanding of various mammalian diseases.

Other assays of interest which may be practiced using the subject method include: genotyping, scanning of known and unknown mutation, gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

The above described applications are merely representations of the numerous different applications for which the subject array and method of use are suited. In certain embodiments, the subject method includes a step of transmitting data from at least one of the detecting and deriving steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

In certain embodiments of the subject methods in an array, the array may typically be read. Reading of the array may be accomplished by illuminating the array and reading the location and intensity of resulting fluorescence at each feature of the array to detect any binding complexes on the surface of the array. For example, a scanner may be used for this purpose which is similar to the AGILENT MICROARRAY SCANNER device available from Agilent Technologies, Santa Clara, Calif. Other suitable apparatus and methods are described in U.S. Pat. Nos. 5,091,652; 5,260,578; 5,296,700; 5,324,633; 5,585,639; 5,760,951; 5,763,870; 6,084,991; 6,222,664; 6,284,465; 6,371,370 6,320,196 and 6,355,934; the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample). The results of the reading (processed or not) may be forwarded (such as by communication) to a remote location if desired, and received there for further use (such as further processing).

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### EXAMPLE 1

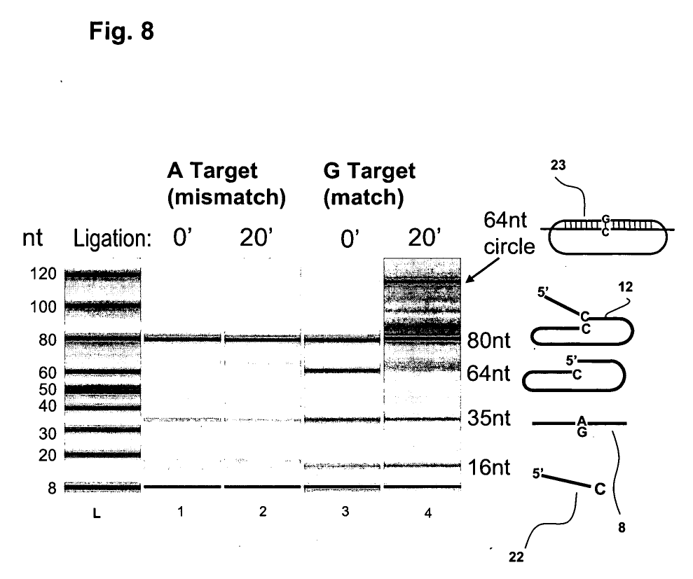

In this example a Q probe corresponding to SEQ ID NO: 1 is preferentially cleaved by human Fen-1 on a target polynucleotide containing the correct allele (SEQ ID NO: 2). Sample data are shown in FIG. 8.

In the first step, the Q probe corresponding to SEQ ID NO: 1 was annealed to target oligonucleotides corresponding to SEQ ID NO: 2 and SEQ ID NO: 3. These components were mixed and incubated in annealing conditions:
15 mM NaCl
10 mM Tris-Cl pH 8.0
1 mM EDTA pH 8.0
10 micromolar Q probe (SEQ ID NO: 1)
10 micromolar target oligonucleotide (SEQ ID NO: 2 or SEQ ID NO: 3)
The annealing reactions were incubated at 98° C. for 2 min; 80° C. for 1 min; 0.2°/sec to 70° C.; 0.1°/sec to 60° C.; 60° C. for 1 min; 0.1°/sec to 50° C.; 50° C. for 1 min; 0.1°/sec to 40° C.; 40° C. for 1 min; 0.1°/sec to 30° C.; 30° C. for 1 min; 0.1°/sec to 20° C.; 20° C. for 1 min; 0.1°/sec to 10° C.; 10° C. for 10 min; 0.1°/sec to 4° C.; 4° C. hold.

Fen-1 cleavage reactions of the Q probe corresponding to SEQ ID NO: 1 on the correct target sequence were performed by combining the following reagents on ice:
3 μL annealed Q probe-target reaction (final concentration of 2 micromolar Q probe)
1 μL 10× Bovine Serum Albumin additive (Trevigen, Inc., Gaithersburg, Md., USA; final concentration 0.1 mg/ml BSA, 5% glycerol)
1.5 μL 10× REC reaction buffer (Trevigen, Inc., Gaithersburg, Md., USA; final concentration 50 mM Tris-HCl (pH 8.0), 10 mM MnCl₂, and 1 mM DTT)
0.35 μL human Fen-1 enzyme (Trevigen, Inc., Gaithersburg, Md., USA.; 1 unit)
9.15 μL deionized water
   The reactions were incubated for 60 min at 30° C. Aliquots were removed for analysis at 0 min, 5 min, 15 min, and 60 minutes.

Samples were analyzed by running 1 μL of the reaction an Agilent Small RNA microfluidic chip, and following the manufacturer's instructions. Electropherograms comparing the cleavage of the Q probe reactions were generated using the Agilent 2100 Expert software.

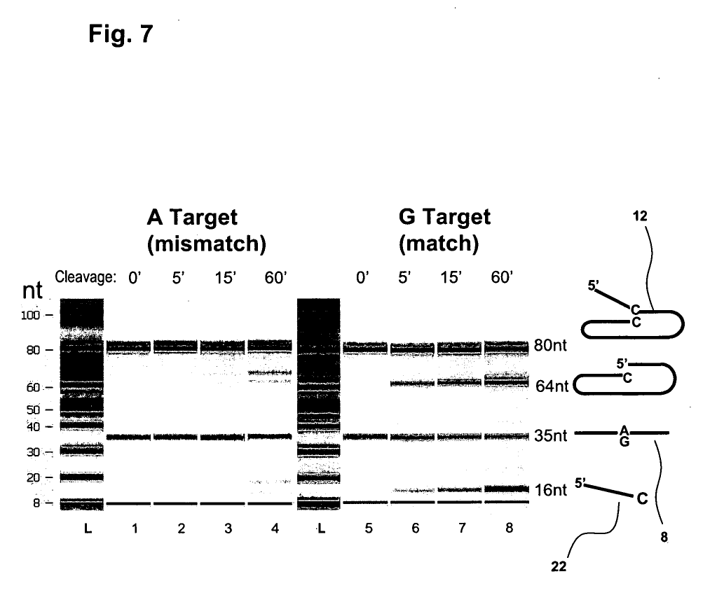

FIG. 7 shows a gel-like image of electropherograms of the FEN cleavage reactions. Diagrams of the oligonucleotides corresponding to the bands in the electropherogram are shown on the right; see also FIG. 2. Lanes marked L show an oligonucleotide ladder showing the mobility of single stranded DNA oligos corresponding to 8, 20, 30, 40, 50, 60, 80, and 100 nucleotides. Lanes 1-4 and 5-8 show Q probe cleavage reactions in the presence of the A target (mismatch target oligo corresponding to SEQ ID NO: 3) or the G target (correct target oligo corresponding to SEQ ID NO: 2). The A and G target oligos are visible as bands at 35 nt. The uncleaved Q probe (corresponding to SEQ ID NO: 1) is visible as a band running at 80 nt. The 2 fragments of the cleaved Q probe are visible as bands running at 16 nt and 64 nt.

Preferential cleavage of the Q probe on the correct target oligo corresponding to SEQ ID NO: 2 is illustrated by the increase in the intensity of the cleavage product bands at 16 and 64 nt in lanes 5-8.

### EXAMPLE 2

This example demonstrates allele-specific ligation of products of a Q probe cleavage reaction. Sample data are shown in FIG. 8. In this example products of the Q probe cleavage reactions described in Example 1 are subjected to ligation conditions.

2.5 μL Fen-1 reaction

0.5 μL 5× T4 ligase buffer (Invitrogen Corp., Carlsbad, Calif., USA)

1 μL T4 DNA ligase, High Concentration (5 units; Invitrogen Corp., Carlsbad, Calif., USA)

1 μL deionized water

The ligation reaction were incubated for 20 min at room temperature (20° C.). Samples were analyzed by running 1 microliter of the reaction on an Agilent Small RNA microfluidic chip, and following the manufacturer's instructions. Electropherograms comparing the cleavage of the Q probe reactions were generated using the Agilent 2100 Expert software.

FIG. 8 shows a gel-like image of electropherograms of the FEN cleavage reactions. Diagrams of the oligonucleotides corresponding to the bands in the electropherogram are shown on the right; see also FIG. 2. The lane marked L shows an oligonucleotide ladder showing the mobility of single stranded DNA oligos corresponding to 8, 20, 30, 40, 50, 60, 80, and 100, and 120 nucleotides. The addition of ligase (Lanes 2 and 4) converts the 64 nt cleavage product to a ssDNA circle that remains annealed to the target fragment (shown as element 23 in FIGS. 2 and 8) that runs near the 120 nt marker. Therefore, allele-specific ligation is shown by the appearance of this slowly-migrating band in the ligation reactions containing the G target oligo corresponding to SEQ ID NO: 2, but not in the ligation reactions containing the A target oligo corresponding to SEQ ID NO: 3.

### EXAMPLE 3

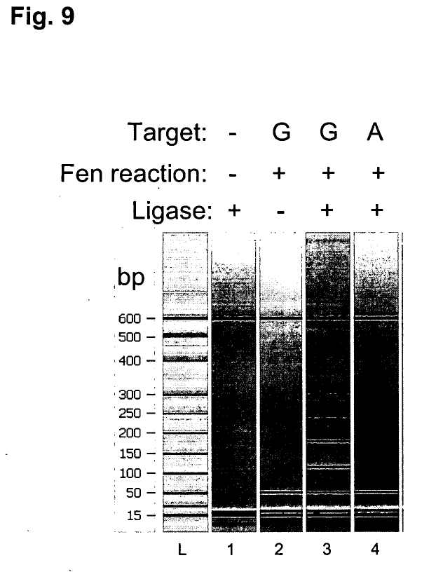

This example demonstrates allele-specific amplification of ligated products of a Q probe cleavage reaction. Sample data are shown in FIG. 9. Ligated Q probe cleavage reactions (as detailed in Examples 1 and 2) are subjected to rolling circle amplification (RCA) conditions.

Phi29 Rolling Circle Amplification Reaction:

1 μL (50 picomoles) RCA primer (SEQ ID NO: 5)

1 μL ligated FEN reaction (2 picomoles)

4 μL deionized water

These components were mixed, heated to 95° C. for 2 min, and cooled on ice. To these reactions were added:

2 μL 10× Phi29 buffer (New England Biolabs, Ipswich, Mass., USA, Ipswich, Mass., USA)

0.24 μL 10 mg/ml Bovine Serum Albumin

0.24 μL 100 mM Dithiothreitol

1 μL dNTPs (10 mM each dA, dT, dG, dC)

13.8 μL deionized water

The reactions were mixed, a 5 μliter aliquot was removed for later analysis, and finally the following was added to each reactions:

1 μLphi29 polymerase (10 units; New England Biolabs, Ipswich, Mass., USA)

The reactions were mixed an incubated for 1 hour at 30° C., followed by a 15 min incubation at 65° C.

In order to ease analysis of the long DNA created by RCA, products of the RCA reaction were combined with an oligo corresponding to SEQ ID NO: 4 and digested with the restriction enzyme BspD1.
BspD1 reaction:
5 μL RCA reaction
0.5 μL of 100 μM SEQ ID NO: 4 oligo in TE buffer
2.5 μL deionized water
   These components were mixed and incubated in annealing conditions:
98° C. for 2 min; 80° C. for 1 min; 0.2°/sec to 70° C.; 0.1°/sec to 60° C.; 60° C. for 1 min; 0.1°/sec to 50° C.; 50° C. for 1 min; 0.1°/sec to 40° C.; 40° C. for 1 min; 0.1/sec to 30° C.; 30° C. for 1 min;
0.1 °/sec to 20° C.; 20° C. for 1 min; 0.1°/sec to 10° C.; 10° C. for 10 min; 0.1°/sec to 4° C.; 4° C. hold.
   Following the annealing step, the reactions were combined with:
1 μL 10× BspD1 buffer (New England Biolabs, Ipswich, Mass., USA)
1 μL (5 units) BspD1 enzyme (New England Biolabs, Ipswich, Mass., USA)

The reactions were mixed an incubated for 2 hours at 37° C., followed by a 20 min incubation at 65° C. 1 μliter of the restriction digest reaction was analyzed on the Agilent Bioanalyzer 2100 using the DNA500 reagents kit, and following the manufacturer's instructions (Agilent Technologies, Santa Clara, Calif., USA).

Sample data are shown as a gel-like image in FIG. 9.

The first lane, labeled “L” shows the electrophoretic mobility of a ladder of double stranded DNA, with sizes indicated.

Lane 1 shows that in the absence of a Fen reaction (which creates the susbtrate for the RCA), there is no amplification.

Lane 2 shows that in the presence of the Fen reaction with cleavage on the correct target (G target reaction containing an oligo corresponding to SEQ ID NO: 2; same reaction as lane 8 in FIG. 7), but in the absence of ligase, there is no amplification. Without ligase, no ssDNA circle is created.

Lane 3 shows a ladder of bands corresponding to partial cleavage of the concatemer created by the RCA of the cleaved and ligated Q probe. Ligation of the Fen reaction creates a 64-nt circle of ssDNA, as shown in Lane 4 of FIG. 8. RCA of this ssDNA circle creates a long concatemer of this sequence. In order to ease visualization, the concatemer is digested with the restriction enzyme BspDI after annealing a short oligonucleotide (SEQ ID NO: 4) to create a site for the enzyme. As the digestion was incomplete, the result is a ladder of bands of 64 nt, 128 nt, 192nt, etc., corresponding to 1, 2, 3, etc., tandem copies of the template circle sequence.

Lane 4 shows that the FEN reaction on the mismatch target (A target, SEQ ID NO: 3) does not support RCA. In this FEN reaction (corresponding to Lane 4 in FIG. 7), even if there is a small amount of cleavage, there is a mismatch inhibiting the ligation of the cleaved Q probe into a circle of ssDNA (see also Lane 2 in FIG. 8). Thus, no circle is created, and the RCA reaction does not create a concatemer of the target sequence.

In summary, this example shows that the presence or absence of amplification of a ligated Q probe reaction can indicate the sequence of the target. Thus, the amplification reaction is allele-specific. In this specific case, only the reaction containing the correct target (G target, SEQ ID NO: 2) supported RCA, whereas the the reaction containing the mismatch target (A target, SEQ ID NO: 3) did not support RCA. This allele-specific amplification method may be useful in cases where the concentration of Q probes is too low to detect cleavage of the Q probe.

### EXAMPLE 4

In this example a fluorescently-labeled Q probe corresponding to SEQ ID NO: 6 is preferentially cleaved by Taq polymerase on a target polynucleotide containing the correct allele (SEQ ID NO: 7). The sequence of this Q probe was designed to detect the state of the SNP with the ID rs2106269. Specifically, in a sample where rs2106269 comprises the T allele (represented by the “A target” corresponding to SEQ ID NO: 7), the Q probe will be cleaved. In a sample where rs2106269 is comprises the C allele (represented by the “G target” corresponding to SEQ ID NO: 8), the Q probe will not be cleaved.

In the first step, the Cy5-labeled Q probe corresponding to SEQ ID NO: 6 was annealed to target oligonucleotides corresponding to SEQ ID NO: 7 and SEQ ID NO: 8. These components were mixed and incubated in annealing conditions:
15 mM NaCl
3 mM Tris-Cl pH 8.0
0.3 mM EDTA pH 8.0
10 μM Cy5-labeled Q probe (SEQ ID NO: 6)
20 μM target oligonucleotide (SEQ ID NO: 7 or SEQ ID NO: 8)

The annealing reactions were incubated at 98° C. for 2 min; 80° C. for 1 min; 0.2°/sec to 70° C.; 0.1°/sec to 60° C.; 60° C. for 1 min; 0.1°/sec to 50° C.; 50° C. for 1 min; 0.1°/sec to 40° C.; 40° C. for 1 min; 0.1°/sec to 30° C.; 30° C. for 1 min; 0.1°/sec to 20° C.; 20° C. for 1 min; 0.1°/sec to 10° C.; 10° C. for 10 min; 0.1°/sec to 4° C.; 4° C. hold.

Preferential cleavage of the Cy5-labeled Q probe corresponding to SEQ ID NO: 6 on the correct target sequence was demonstrated by combining the following reagents in a 40 microliter reaction volume:
25 mM Tris-Cl pH 9.0
5 mM MgCl₂
50 mM KCl
2 mM Dithiothreitiol
2 units Taq polymerase (Invitrogen Corporation, Carlsbad, Calif.)
Deionized water
250 nanomolar Q-probe-target complex

The reaction was incubated at 75° C. for 10 min, and the reaction was stopped by adding EDTA to 6.7 mM and immediately freezing tubes on dry ice before analysis. Samples were analyzed by running 1 microliter of the stopped reaction an an Agilent Small RNA microfluidic chip, and following the manufacturer's instructions (Agilent Technologies, Santa Clara, Calif., USA). Electropherograms comparing the cleavage of the Q probe reactions were generated using the Agilent 2100 Expert software (Agilent Technologies, Santa Clara, Calif., USA).

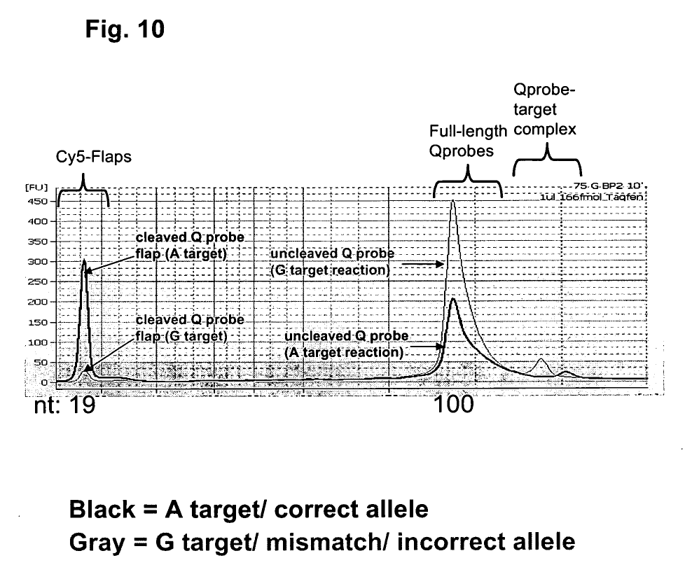

A sample electropherogram showing the preferential cleavage of the Q probe corresponding to SEQ ID NO: 6 on the A target (SEQ ID NO: 7) is shown in FIG. 10. In this electropherogram, only the Cy5 labeled oligonucleotides are visible; thus the target polynucleotides corresponding to SEQ ID NO: 7 and SEQ ID NO: 8, which are present in the cleavage reactions, are not seen. Preferential cleavage of the Q probe in the FEN reaction containing the A target (SEQ ID NO: 7) is demonstrated by the smaller peak near 100 nt corresponding to the uncleaved Q probe, and the larger peak near 19 nt corresponding to the fluorescently labeled, cleaved flap oligonucleotide.

### SEQUENCES

An example of a Q probe sequence is shown in SEQ ID NO: 1

5′ TCACTATAGGGAGACCGGAATCGATTTTCTTGTTCAGGATAATGATT
GCCTACGATGATTTTTTACACTATAGAATACAC 3′

G (match) target for the Q probe of SEQ ID NO: 1 is shown in SEQ ID NO: 2:

5′ TTTGCGCTCGATTCCGTGTATTCTATAGTGTTTT 3′

A (mismatch) target for the Q probe of SEQ ID NO: 1 is shown in SEQ ID NO: 3:

5′ TTTGCGCTCGATTCCATGTATTCTATAGTGTTTT 3′

SEQ ID NO: 4

5′- TAC ACG GAA TCG ATT TTC TTG TTC -3′

SEQ ID NO: 5

5′ CGT AGG CAA TCA TTA TCC TG 3′

The sequence of a Cy5-labeled Q probe targeting SNP ID rs2106269 is shown in SEQ ID NO: 6:

5′ (Cy5)
GCGGTCTGCTGAGCGGTCTGGGGGAGTAACATTTAGATCTGCACGATAAC
GGTAGAAAGCTTCTGCAGGATATCTGGATCCACAGCTCTAGAGAAGCAA
T 3′

The A (match) target for the Q probe of SEQ ID NO: 6 is shown in SEQ ID NO: 7:

5′- TTA TGT TAC TCC CCC AAT TGC TTC TCT AGA GCT
GTT -3′

The G (mismatch) target for the Q probe of SEQ ID NO: 6 is shown in SEQ ID NO: 8:

5′- TTA TGT TAC TCC CCC AGT TGC TTC TCT AGA GCT
GTT -3′

What is claimed is:

1. A method comprising:
a) contacting a plurality of Q probes with a nucleic acid sample comprising a target polynucleotide under hybridization conditions to form a plurality of flap endonuclease substrates each comprising a Q probe and a site in said target polynucleotide;
b) contacting said plurality of flap endonuclease substrates with a flap endonuclease under cleavage conditions to produce cleavage products, wherein each of said Q probes of said flap endonuclease substrates is cleaved to produce cleavage products that include at least a first fragment that is linear and free in solution and a second fragment that is hybridized with said site in said target polynucleotide; and
c) detecting at least one of said cleavage products.

2. The method of claim 1, wherein said plurality of flap endonuclease substrates are Tₘ-matched.

3. The method of claim 1, wherein said detecting comprises detecting said first fragment.

4. The method of claim 1, wherein said detecting comprises detecting said second fragment.

5. The composition of claim 4, wherein said detecting comprises ligating said first fragment to an oligonucleotide on a solid support.

6. The method of claim 1, wherein said detecting comprises hybridization to an array.

7. The method of claim 1, wherein said method comprises
i) denaturing said complexes after said contacting step b) and prior to said detecting step c); and
ii) repeating step a) and step b) to generate additional cleavage products prior to said detecting step c).

8. The method of claim 1, wherein said hybridization conditions and said cleavage conditions comprise a temperature that is higher than a predicted Tₘ of a duplex region in said flap endonuclease substrates.

9. The method of claim 3, wherein said temperature is in a range between 60 and 90 degree Celsius.

10. The method of claim 1, wherein said flap endonuclease is thermostable.

11. The method of claim 1, wherein a molar ratio of said Q probes to said target polynucleotide is less than 1,000.

12. The method of claim 1, wherein the length of said first or second fragment varies, wherein said length uniquely identifies which Q probe that has been cleaved.

13. The method of claim 1, wherein said Q probes are greater than 100 nucleotides in length.

14. The method of claim 1, wherein said detecting step c) comprises:
i) ligating the ends of said first fragment hybridized to said site to produced an intramolecularly ligated circular product; and
ii) detecting said intramolecularly ligated circular product.

15. The method of claim 1, wherein said detecting step c) comprises:
i) amplifying said cleavage products to produce amplified products; and
ii) detecting said amplified products.

16. The method of claim 1, wherein said flap endonuclease substrates comprise at least one duplex region of 9 or fewer base pairs.

17. The method of claim 1, wherein said method comprises degrading said target polynucleotide prior to detecting at least one of said cleavage products.

18. The method of claim 1, wherein sites on said target polynucleotide to which said Q probes bind are sites of a single-nucleotide polymorphism.

19. A composition comprising a plurality of Q probes, wherein hybridization of said Q probes to a plurality of target polynucleotides forms a plurality of flap endonuclease substrates.

20. A kit for analyzing target polynucleotides according to the method of claim 1, comprising:
a) a plurality of Q probes, wherein hybridization of said Q probes to a plurality of target polynucleotides forms a plurality of flap endonuclease substrates.
b) a flap endonuclease.


SEQUENCE LISTING (8 sequences)

SEQ ID NO: 1 - DNA, 80 bp, Artificial Sequence
  synthetic oligonucleotide
tcactatagggagaccggaatcgattttcttgttcaggataatgattgcctacgatgattttttacactatagaatacac

SEQ ID NO: 2 - DNA, 34 bp, Artificial Sequence
  synthetic oligonucleotide
tttgcgctcgattccgtgtattctatagtgtttt

SEQ ID NO: 3 - DNA, 34 bp, Artificial Sequence
  synthetic oligonucleotide
tttgcgctcgattccatgtattctatagtgtttt

SEQ ID NO: 4 - DNA, 24 bp, Artificial Sequence
  synthetic oligonucleotide
tacacggaatcgattttcttgttc

SEQ ID NO: 5 - DNA, 20 bp, Artificial Sequence
  synthetic oligonucleotide
cgtaggcaatcattatcctg

SEQ ID NO: 6 - DNA, 100 bp, Artificial Sequence
  synthetic oligonucleotide
gcggtctgctgagcggtctgggggagtaacatttagatctgcacgataacggtagaaagcttctgcaggatatctggatccacagctctagagaagcaat

SEQ ID NO: 7 - DNA, 36 bp, Artificial Sequence
  synthetic oligonucleotide
ttatgttactcccccaattgcttctctagagctgtt

SEQ ID NO: 8 - DNA, 36 bp, Artificial Sequence
  synthetic oligonucleotide
ttatgttactcccccagttgcttctctagagctgtt